(19) United States
(12) Patent Application Publication
(10) Pub. No.: US 2022/0110548 A1
(43) Pub. Date: Apr. 14, 2022

(54) MOVEMENT MONITORING SYSTEM

(71) Applicant: WISCONSIN ALUMNI RESEARCH FOUNDATION, MADISON, WI (US)

(72) Inventors: ROBERT RADWIN, WAUNAKEE, WI (US); YIN LI, MADISON, WI (US); RUNYU GREENE, MADISON, WI (US); FANGZHOU MU, MADISON, WI (US); YU HEN HU, MIDDLETON, WI (US)

(73) Assignee: WISCONSIN ALUMNI RESEARCH FOUNDATION, MADISON, WI (US)

(21) Appl. No.: 17/497,676

(22) Filed: Oct. 8, 2021

Related U.S. Application Data
(60) Provisional application No. 63/089,714, filed on Oct. 9, 2020.

(51) Int. Cl. A61B 5/11; A61B 5/00; G16H 50/30

(52) U.S. Cl. CPC A61B 5/1128; A61B 5/0004; A61B 5/1113; A61B 2560/02; G16H 50/30; A61B 2503/20; A61B 5/7267

(57) ABSTRACT

A monitoring or tracking system may include an input port and a controller in communication with the input port. The input port may receive data from a data recorder. The data recorder is optionally part of the monitoring system and in some cases includes at least part of the controller. The controller may be configured to receive data via the input port, the data being related to a subject lifting an object. Using the data, the controller may locate body parts of the subject while the subject is lifting the object and monitor body movements of the subject during the lift. Further, the controller may be configured to determine a value related to a load of the object based on the body movements monitored. The controller may output via the output port the value determined and/or lift assessment information for the subject.

### CROSS REFERENCE TO RELATED APPLICATIONS

This application claims the benefit of U.S. Provisional Patent Application No. 63/089,714, filed on Oct. 9, 2020, the disclosure of which is incorporated herein by reference in its entirety for any and all purposes.

### STATEMENT REGARDING FEDERALLY SPONSORED RESEARCH OR DEVELOPMENT

This invention was made with government support under R01 OH011024 awarded by the Center for Disease Control and Prevention. The government has certain rights in the invention.

### TECHNICAL FIELD

The present disclosure pertains to monitoring systems and assessment tools, and the like. More particularly, the present disclosure pertains to video analysis monitoring systems and systems for assessing risks associated with movement and exertions.

### BACKGROUND

A variety of approaches and systems have been developed to monitor physical stress on a subject. Such monitoring approaches and systems may require manual observations and recordings, cumbersome wearable instruments, complex linkage algorithms, and/or complex three-dimensional (3D) tracking. More specifically, the developed monitoring approaches and systems may require detailed manual measurements, manual observations over a long period of time, observer training, sensors on a subject, and/or complex recording devices. Of the known approaches and systems for monitoring physical stress on a subject, each has certain advantages and disadvantages.

### SUMMARY

This disclosure is directed to several alternative designs for, devices of, and methods of using monitoring systems and assessment tools. Although it is noted that monitoring approaches and systems are known, there exists a need for improvement on those approaches and systems.

Accordingly, one illustrative instance of the disclosure may include a subject monitoring system. The subject monitoring system may include an input port and a controller in communication with the input port. The input port may receive data related to a subject lifting an object. The controller may be configured to monitor body movements of the subject lifting the object using the data and determine a value related to a load of the object based on the body movements monitored.

Alternatively or additionally to any of the embodiments above, the controller may be further configured to locate a plurality of body parts of the subject using the data. The monitoring body movements of the subject may include identifying coordinates of one or more body parts of the located plurality of body parts at one or more time instances while the subject is lifting the object.

Alternatively or additionally to any of the embodiments above, the plurality of body parts of the subject may include two or more of the following body parts: a head of the subject, a left shoulder of the subject, a right shoulder of the subject, a left elbow of the subject, a right elbow of the subject, a left wrist of the subject, a right wrist of the subject, a left hip of the subject, a right hip of the subject, a left knee of the subject, a right knee of the subject, a left ankle of the subject, and a right ankle of the subject.

Alternatively or additionally to any of the embodiments above, the monitoring body movements of the subject may include determining body kinematics of the subject lifting the object.

Alternatively or additionally to any of the embodiments above, the determined body kinematics of the subject may include one or both of a velocity of a body part of the subject while the subject is lifting the object and an acceleration of a body part of the subject while the subject is lifting the object.

Alternatively or additionally to any of the embodiments above, the monitoring body movements of the subject may include determining a posture of the subject while the subject is lifting the object.

Alternatively or additionally to any of the embodiments above, the value related to a load of the object based on the body movements monitored may be an estimated value of the load of the object.

Alternatively or additionally to any of the embodiments above, the value related to a load of the object based on the body movements monitored may be a category indicative of a relative value of the load of the object.

Alternatively or additionally to any of the embodiments above, the value related to a load of the object based on the body movements monitored may be a lifting index value, the lifting index value is a load of the object estimated based on the body movements monitored divided by a recommended weight limit (RWL) for the subject lifting the object.

Alternatively or additionally to any of the embodiments above, the controller may be further configured to determine the RWL based on the data.

Alternatively or additionally to any of the embodiments above, the controller may be further configured to train a neural network model of the subject using the data, and the value related to a load of the object may be determined based on the neural network model of the subject and the monitored body movements.

Another illustrative instance of the disclosure may include a computer readable medium having stored thereon in a non-transitory state program code for use by a computing device, the program code may cause the computing device to execute a method for determining a value related to a load of an object lifted by a subject. The method may include locating one or more body parts of the subject using data related to the subject lifting the object, monitoring one or more body parts of the subject while the subject is lifting the object, and determining a value related to a load of the object based on the one or more body parts monitored.

Alternatively or additionally to any of the embodiments above, the monitoring of the one or more body parts of the subject may include determining body kinematics of the subject lifting the object.

Alternatively or additionally to any of the embodiments above, the monitoring the one or more body parts of the subject may include determining a posture of the subject.

Alternatively or additionally to any of the embodiments above, the determining the value related to the load of the object may include determining an estimate of the load of the object based on the body movements monitored.

Alternatively or additionally to any of the embodiments above, the method may further include determining a recommended weight limit (RWL) for the subject lifting the object using the data related to the subject lifting the object, and the determining the value related to the load of the object may include determining a value of a lifting index using the estimate of the load of the object and the RWL.

Another illustrative instances of the disclosure may include a method of determining a load of an object lifted by a subject. The method may include monitoring one or more body parts of a subject while the subject is lifting an object, determining kinematics of at least one body part of the subject while the subject is lifting the object based on the monitoring of the one or more body parts of the subject, and determining a value related to a load of the object based on the determined kinematics of the at least one body part.

Alternatively or additionally to any of the embodiments above, the method may further include determining a posture of the subject lifting the object based on the monitoring of the one or more body parts of the subject.

Alternatively or additionally to any of the embodiments above, the determining the value related to the load of the object may include determining an estimate of the load of the object.

Alternatively or additionally to any of the embodiments above, the monitoring of the one or more body parts of the subject may include applying a bounding box around the subject in a video of the subject lifting the object.

The above summary of some example embodiments is not intended to describe each disclosed embodiment or every implementation of the disclosure.

### BRIEF DESCRIPTION OF THE DRAWINGS

The disclosure may be more completely understood in consideration of the following detailed description of various embodiments in connection with the accompanying drawings, in which:

FIG. 1 is a schematic box diagram of an illustrative monitoring or tracking system;

FIG. 2 is a schematic box diagram depicting an illustrative flow of data in a monitoring system;

FIG. 3 is a schematic flow diagram of an illustrative method of determining a value related to a load of an object lifted by a subject;

FIG. 4 is a schematic flow diagram of an illustrative method of determining a value related to a load of an object lifted by a subject; and

FIG. 5 is a schematic diagram of an illustrative technique for determining a value related to a load of an objected lifted by a subject.

While the disclosure is amenable to various modifications and alternative forms, specifics thereof have been shown by way of example in the drawings and will be described in detail. It should be understood, however, that the intention is not to limit aspects of the claimed disclosure to the particular embodiments described. On the contrary, the intention is to cover all modifications, equivalents, and alternatives falling within the spirit and scope of the claimed disclosure.

### DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the claimed disclosure. The illustrative embodiments depicted are intended only as exemplary. Selected features of any illustrative embodiment may be incorporated into an additional embodiment unless clearly stated to the contrary.

Physical exertion is a part of many jobs. For example, manufacturing and industrial jobs may require workers to perform manual lifting tasks (e.g., an event of interest or predetermined task during which a subject picks up, sets down, raises, lowers, and/or otherwise moves an object). In some cases, these manual lifting tasks may be repeated throughout the day. Additionally, people perform lifting tasks and/or activities regularly throughout the day. Assessing lifts, movements, and/or exertions by workers and/or other people while performing tasks required by manufacturing and/or industrial jobs and/or movements of people in other jobs or activities may facilitate reducing injuries by identifying movement and/or activities that may put a person at risk for injury.

Repetitive work and/or activities (e.g., manual work or other work and/or activities) may be associated with muscle fatigue, back strain, injury, and/or other pain as a result of stress and/or strain on a person's body. As such, repetitive work and activities (e.g., lifting, etc.) have been studied extensively. For example, studies have analyzed what is a proper posture that reduces physical injury risk to a minimum while performing certain tasks and also, how movement cycles (e.g., work cycles) and associated parameters (e.g., a load, a horizontal location of the origin and destination of the motion (e.g., a lift motion or other motion), a vertical location of the origin and destination of the motion, a distance of the motion, a frequency of the motion, a duration of the movement, a twisting angle during the motion, a coupling with an object, etc.) relate to injury risk. Other parameters associated with movement cycles that may contribute to injury risk may include the velocity and acceleration of movement of the subject (e.g., velocity and/or acceleration of trunk movement and/or other suitable velocity and/or acceleration of movement), the angle of a body of the subject (e.g., a trunk angle or other suitable angles of the body), and/or an object moved at an origin and/or destination of movement. Some of these parameters may be used to identify a person's risk for an injury during a task based on guidelines such as the revised National Institute for Occupational Safety and Health (NIOSH) lifting equation (RNLE) or the American Conference of Governmental Industrial Hygienists (ACGIH) Threshold Limit Value (TLV) for manual lifting, among others. Additionally, some of these parameters have been shown to be indicative of injury risk (e.g., risk of lower back pain (LBP) or lower back disorders (LBD), etc.), but are not typically utilized in identifying a person's risk for an injury during a task due to it being difficult to obtain consistent and accurate measurements of the parameters.

In order to control effects of repetitive work on the body, quantification of parameters such as posture assumed by the body while performing a task, the origin and/or destination of objects lifted during a task, duration of the task, position assumed during the task, a trunk angle assumed by the body while performing a task, kinematics of the body during the task, frequency of the task, and a load (e.g., a weight) of an object being lifted, among other parameters, may facilitate evaluating an injury risk for a worker performing the task. A limitation, however, of identifying postures, trunk angles, trunk angle kinematics, the origin and destination of movement or moved objects, a load of an object handled (e.g., lifted), and/or analyzing movement cycles is that it can be difficult to extract parameter measurements from an observed scene during a task.

In some cases, wearable equipment may be used to obtain and/or record values of parameters in an observed scene during a task. Although the wearable equipment may provide accurate sensor data, such wearable equipment may require a considerable set-up process, may be cumbersome, and may impede the wearer's movements and/or load the wearer's body, and as a result, may affect performance of the wearer such that the observed movements are not natural movements made by the wearer when performing the observed task. Furthermore, it has been difficult to identify an actual context of signals obtained from wearable instrument data alone.

An example of commonly used wearable equipment is a lumbar motion monitor (LMM), which may be used to obtain and/or record values of parameters relating to movement of a subject performing a task. The LMM is an exoskeleton of the spine that may be attached to the shoulder and hips of a subject using a harness. Based on this configuration, the LMM may provide reliable measurements of a position, velocity, and acceleration of the subject's trunk while the subject is performing a task. However, similar to other wearable equipment, the LMM may be costly, require a considerable set-up/training process, may impose interruptions to the wearer's regular tasks, and may be difficult to identify the context of signals, etc. The burdens of wearing the LMM, or other wearable instruments, and lack of other options for accurately measuring a trunk angle and/or trunk kinematics of a subject performing a task has led to safety organizations omitting trunk angle and/or trunk kinematics from injury risk assessments despite trunk angle and trunk kinematics being associated with work-related low-back disorders.

Observing a scene without directly affecting movement of the person performing the task may be accomplished by recording the person's movements using video. In some cases, complex 3D video equipment and measurement sensors may be used to capture video of a person performing a task.

Recorded video (e.g., image data of the recorded video) may be processed in one or more manners to identify and/or extract parameters from the recorded scene. Some approaches for processing the image data may include recognizing a body of the observed person and each limb associated with the body in the image data. Once the body and limbs are recognized, motion parameters of the observed person may be analyzed. Identifying and tracking the body and the limbs of an observed person, however, may be difficult and may require complex algorithms and classification schemes. Such difficulties in identifying the body and limbs extending therefrom stem from the various shapes bodies and limbs may take and a limited number of distinguishable features for representing the body and limbs as the observed person changes configurations (e.g., postures) while performing a task.

Video (e.g., image data recorded with virtually any digital camera) of a subject performing a task may be analyzed with an approach that does not require complex classification systems, where the approach results in using less computing power and taking less time for analyses than the more complex and/or cumbersome approaches discussed above. In some cases, this approach may be, or may be embodied in, a marker-less tracking system. In one example, the marker-less tracking system may identify feature points, a contour, and/or a portion of a subject (e.g., a body of interest, a person, an animal, a machine, and/or other subject) and determine parameter measurements from the subject in one or more frames of the video (e.g., a width dimension and/or a height dimension of the subject, a location of hands, feet, a head, elbows, wrists, ankles, hips, knees and/or other body features of a subject, a distance between hands and feet of the subject, when the subject is beginning and/or ending a task, and/or other suitable parameter values). In some cases, a bounding box may be placed around the subject and the dimension of the bounding box may be used for determining one or more parameter values and/or position assessment values relative to the subject. In another example, three-dimensional coordinates of features points of the subject performing the task may be determined, without a complex 3D tracking system, once the subject in the video is identified.

Although other computer vision systems are contemplated, example computer vision systems configured to monitor and/or track subjects and/or features of subjects in video are discussed and described in: U.S. Patent Application Ser. No. 62/932,802 filed on Nov. 8, 2019, titled MOVEMENT MONITORING SYSTEM, which is hereby incorporated by reference in its entirety for any and all purposes; U.S. Patent Application Publication No. 2020/0279102 A1 filed on May 15, 2020, titled MOVEMENT MONITORING SYSTEM, which is hereby incorporated by reference in its entirety for any and all purposes; Greene, R. L., Hu, Y. H., Difranco, N., Wang, X., Lu, M. L., Bao, S., Lin, J. H., & Radwin, R. G. (2019), “Predicting Sagittal Plane Lifting Postures from Image Bounding Box Dimensions”, Human factors, 61(1), 64-77, which is hereby incorporated by reference in its entirety for any and all purposes; and Wang, X., Hu, Y. H., Lu, M. L., & Radwin, R. G. (2019), “The accuracy of a 2d video-based lifting monitor”, Ergonomics, 62(8), 1043-1054, which is hereby incorporated by reference in its entirety for any and all purposes.

The data obtained from the above noted approaches or techniques for observing and analyzing movement of a subject and/or other suitable data related to a subject may be utilized for analyzing positions and/or movements of the subject and providing position and/or risk assessment information of the subject using lifting guidelines, including, but not limited to, the RNLE and the ACGIH TLV for manual lifting. Although the NIOSH (e.g., the RNLE) and ACGIH equations are discussed herein, other equations and/or analyses may be performed when doing a risk assessment of movement based on observed data of a subject performing a task and/or otherwise moving, including analyses that assess injury risks based on values related to position information and kinematics of a subject's features during a lift.

The RNLE is a tool used by safety professionals to assess manual material handling jobs and provides an empirical method for computing a weight limit for manual lifting. The RNLE takes into account measurable parameters including a vertical and horizontal location of a lifted object relative to a body of a subject, duration and frequency of the task, a distance the object is moved vertically during the task, a coupling or quality of the subject's grip on the object lifted/carried in the task, and an asymmetry angle or twisting required during the task. A primary product of the RNLE is a Recommended Weight Limit (RWL) for the task. The RWL prescribes a maximum acceptable weight (e.g., a load) that nearly all healthy employees could lift over the course of an eight (8) hour shift without increasing a risk of musculoskeletal disorders (MSD) to the lower back. A Lifting Index (LI) may be developed from the RWL to provide an estimate of a level of physical stress on the subject and MSD risk associated with the task.

The RNLE for a single lift is:

LC×HM×VM×DM×AM×FM×CM=RWL (1)

LC, in equation (1), is a load constant of typically 51 pounds, HM is a horizontal multiplier that represents a horizontal distance between a held load and a subject's spine, VM is a vertical multiplier that represents a vertical height of a lift, DM is a distance multiplier that represents a total distance a load is moved, AM is an asymmetric multiplier that represents an angle between a subject's sagittal plane and a plane of asymmetry (the asymmetry plane may be the vertical plane that intersects the midpoint between the ankles and the midpoint between the knuckles at an asymmetric location), FM is a frequency multiplier that represents a frequency rate of a task, and CM is a coupling multiplier that represents a type of coupling or grip a subject may have on a load. The Lifting Index (LI) is defined as:

(Weight lifted)/(RWL)=LI (2)

The “weight lifted” in equation (2) is a load of the object lifted during a lift. In some cases, the “weight lifted” may be the average weight (e.g., load) of objects lifted during the task or, alternatively, a maximum weight of the objects lifted during the task. The “weight lifted” is often difficult to determine due to a need to weigh all of the objects lifted by a subject. The NIOSH Lifting Equation is described in greater detail in Waters, Thomas R. et al., “Revised NIOSH equation for the design and evaluation of manual lifting tasks”, Ergonomics, volume 36, No. 7, pages 749-776 (1993), which is hereby incorporated by reference in its entirety for any and all purposes.

The ACGIH TLVs are tools used by safety professionals to represent recommended workplace lifting conditions under which it is believed nearly all workers may be repeatedly exposed day after day without developing work-related low back and/or shoulder disorders associated with repetitive lifting tasks. The ACGIH TLVs take into account a vertical and a horizontal location of a lifted object relative to a body of a subject, along with a duration and frequency of the task. The ACGIH TLVs provide three tables with weight limits for two-handed mono-lifting tasks within thirty (30) degrees of the sagittal (i.e., neutral forward) plane. “Mono-lifting” tasks are tasks in which loads are similar and repeated throughout a work day. It may be advantageous to know a load of an object lifted by a subject to ensure the weight limits are not exceeded, but it has been difficult to estimate loads of objects lifted without manually weighing each object.

This disclosure discloses approaches for analyzing data related to a subject performing a task. The data related to a subject performing a task may be obtained through one of the above noted task observation approaches or techniques and/or through one or more other suitable approaches or techniques. As such, although the data analyzing approaches or techniques described herein may be primarily described with respect to and/or in conjunction with data obtained from a marker-less tracking system, the data analyzing approaches or techniques described herein may be utilized to analyze data obtained with other subject observation approaches or techniques.

As discussed above, a lifting load (e.g., a weight of an object lifted) may be an important factor for lifting exposure analysis and risk assessment for a subject performing a lift. For example, the RNLE, which was discussed above in detail, requires an input of a lifting load to calculate the lifting index used for assessing risk of a lower back injury during a lift. Other suitable lifting exposure analysis equations, risk assessment equations, and/or other algorithms that may utilize a lifting load as an input are contemplated. This disclosure discloses an approach for analyzing data to determine in an automated manner values related to the lifting load of an object (e.g., values of the lifting load, values of a category indicating a relative weight/load (e.g., light, medium, heavy, etc.) of the lifting load, percent values of a load relative to maximum load for a subject, etc.) by analyzing body part movements without a need to manually weigh each object lifted.

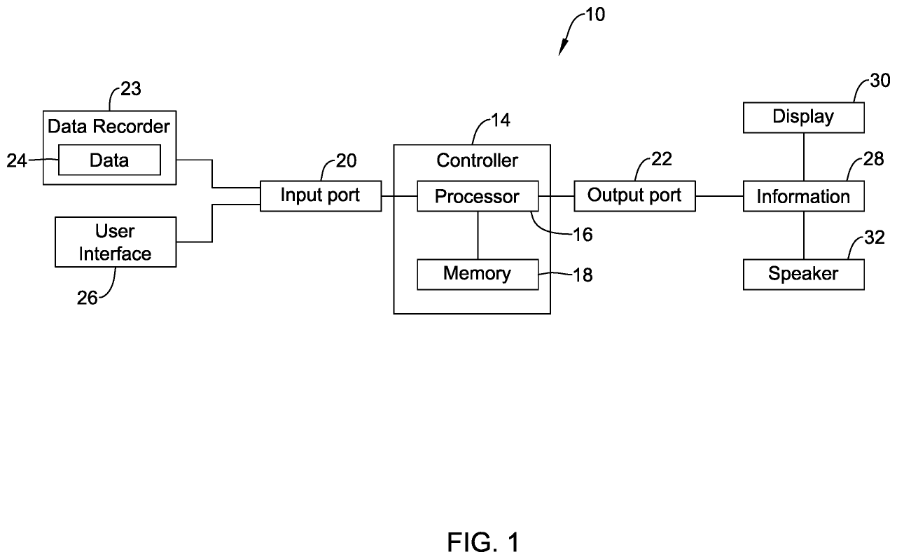

Turning to the Figures, FIG. 1 depicts a schematic box diagram of a monitoring or tracking system 10 (e.g., a marker-less subject monitoring or tracking system). The tracking system 10, as depicted in FIG. 1, may include a controller 14 having a processor 16 (e.g., a microprocessor, microcontroller, or other processor) and memory 18. In some cases, the controller 14 may include a timer (not shown). The timer may be integral to the processor 16 or may be provided as a separate component.

The tracking system 10 may include an input port 20 and an output port 22 configured to communicate with one or more components of or in communication with the controller 14 and/or with one or more remote devices over a network (e.g., a single network or two or more networks). The input port 20 may be configured to receive inputs such as data 24 (e.g., digital data and/or other data from a data capturing device and/or manually obtained and/or inputted data) from a data recorder 23 (e.g., an image capturing device, a sensor system, a computing device receiving manual entry of data, and/or other suitable data recorder), signals from a user interface 26 (e.g., a display, keypad, touch screen, mouse, stylus, microphone, and/or other user interface device), communication signals, and/or other suitable inputs. The output port 22 may be configured to output information 28 (e.g., alerts, alarms, analysis of processed video, and/or other information), control signals, and/or communication signals to a display 30 (a light, LCD, LED, touch screen, and/or other display), a speaker 32, and/or other suitable electrical devices.

In some cases, the display 30 and/or the speaker 32, when included, may be components of the user interface 26, but this is not required, and the display 30 and/or the speaker 32 may be, or may be part of, a device or component separate from the user interface 26. Further, the user interface 26, the display 30, and/or the speaker 32 may be part of the data recording device or system 23 configured to record data 24 related a subject performing a task, but this is not required.

The input port 20 and/or the output port 22 may be configured to receive and/or send information and/or communication signals using one or more protocols. For example, the input port 20 and/or the output port 22 may communicate with other devices or components using a wired or wireless connection, ZigBee, Bluetooth, WiFi, IrDA, dedicated short range communication (DSRC), Near-Field Communications (NFC), EnOcean, and/or any other suitable common or proprietary wired or wireless protocol, as desired.

In some cases, the data recorder 23 may be configured to record data related to a subject performing a task and may provide the data 24 to the controller 14 for analysis. The data recorder 23 may include or may be separate from the user interface 26, the display 30, and/or the speaker 32. One or more of the data recorders 23, the user interface 26, the display 30, the speaker 32, and/or other suitable components may be part of the tracking system 10 or separate from the tracking systems 10. When one or more of the data recorder 23, the user interface 26, the display 30, and/or the speaker 32 are part of the tracking system 10, the features of the tracking system 10 may be in a single device (e.g., two or more of the data recorder 23, the controller 14, the user interface 26, the display 30, the speaker 32, and/or suitable components may all be in a single device) or may be in multiple devices (e.g., the data recorder 23 may be a component that is separate from the display 30, but this is not required). In some cases, the tracking system 10 may exist substantially entirely in a computer readable medium (e.g., memory 18, other memory, or other computer readable medium) having instructions (e.g., a control algorithm or other instructions) stored in a non-transitory state thereon that are executable by a processor (e.g., the processor 16 or other processor) to cause the processor to perform the instructions.

The memory 18 of the controller 14 may be in communication with the processor 16. The memory 18 may be used to store any desired information, such as the aforementioned tracking system 10 (e.g., a control algorithm), recorded data 24 (e.g., video and/or other suitable recorded data), parameters values (e.g., frequency, speed, acceleration, etc.) extracted from data, thresholds, equations for use in analyses (e.g., the RNLE, the Lifting Index, the ACGIH TLV for Manual Lifting, etc.), and the like. The memory 18 may be any suitable type of storage device including, but not limited to, RAM, ROM, EEPROM, flash memory, a hard drive, and/or the like. In some cases, the processor 16 may store information within the memory 18, and may subsequently retrieve the stored information from the memory 18.

As discussed with respect to FIG. 1, the monitoring or tracking system 10 may take on one or more of a variety of forms and the monitoring or tracking system 10 may include or may be located on one or more electronic devices. In some cases, the data recorder 23 used with or of the monitoring or tracking system 10 may process the data 24 thereon. Alternatively, or in addition, the data recorder 23 may send, via a wired connection or wireless connection, at least part of the recorded data or at least partially processed data to a computing device (e.g., a laptop, desktop computer, server, a smart phone, a tablet computer, and/or other computer device) included in or separate from the monitoring or tracking system 10 for processing.

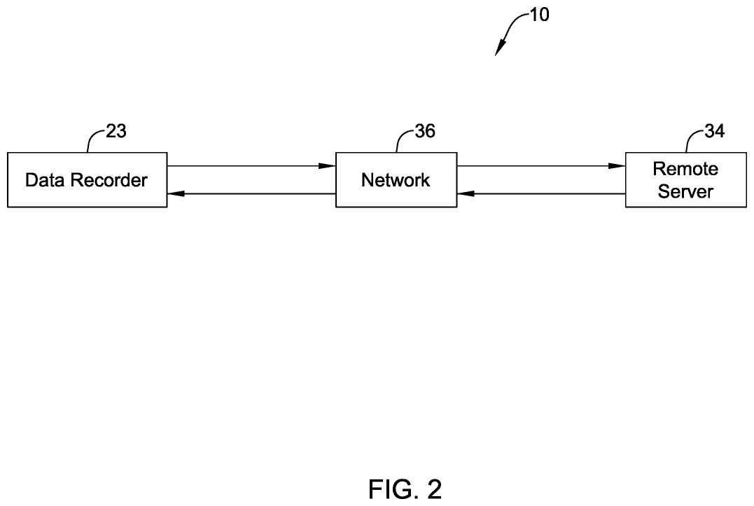

FIG. 2 depicts a schematic box diagram of the monitoring or tracking system 10 having the data recorder 23 connected to a remote server 34 (e.g., a computing device such as a web server or other server) through a network 36. When so configured, the data recorder 23 may send recorded data to the remote server 34 over the network 36 for processing. Alternatively, or in addition, the data recorder 23 and/or an intermediary device (not necessarily shown) between the data recorder 23 and the remote server 34 may process a portion of the data and send the partially processed data to the remote server 34 for further processing and/or analyses. The remote server 34 may process the data and send the processed data and/or results of the processing of the data (e.g., a risk assessment, a recommended weight limit (RWL), a load of an object lifted, a lifting index (LI), etc.) back to the data recorder 23, send the results to other electronic devices, save the results in a database, and/or perform one or more other actions.

The remote server 34 may be any suitable computing device configured to process and/or analyze data and communicate with a remote device (e.g., the data recorder 23 or other remote device). In some cases, the remote server 34 may have more processing power than the data recorder 23 and thus, may be more suitable than the data recorder 23 for analyzing the data recorded by the data recorder 23, but this is not always the case.

The network 36 may include a single network or multiple networks to facilitate communication among devices connected to the network 36. For example, the network 36 may include a wired network, a wireless local area network (LAN), a wide area network (WAN) (e.g., the Internet), and/or one or more other networks. In some cases, to communicate on the wireless LAN, the output port 22 may include a wireless access point and/or a network host device and in other cases, the output port 22 may communicate with a wireless access point and/or a network access point that is separate from the output port 22 and/or the data recorder 23. Further, the wireless LAN may include a local domain name server (DNS), but this is not required for all embodiments. In some cases, the wireless LAN may be an ad hoc wireless network, but this is not required.

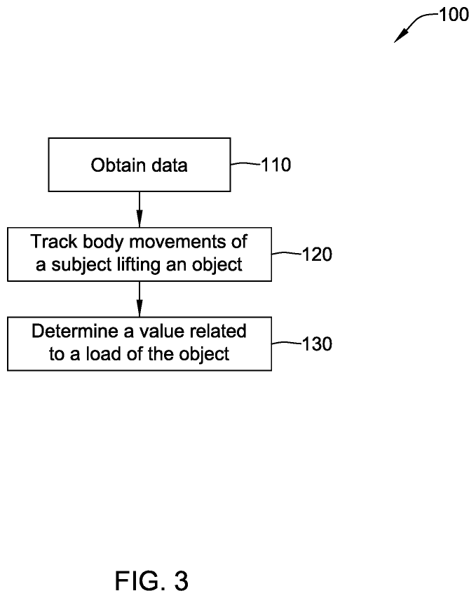

FIG. 3 depicts a schematic overview of an approach 100 for identifying, analyzing, and/or tracking movement of a subject to determine a load of an object based on received data. The approach 100 may be implemented using the tracking system 10, where instructions to perform the elements of the approach 100 may be stored in the memory 18 and executed by the processor 16. Additionally or alternatively, other suitable monitoring and/or tracking systems may be utilized to implement the approach 100.

The approach 100 may include receiving 110 data (e.g., the data 24 and/or other suitable data) from a data source (e.g., the data recorder 23 and/or other suitable source). In some instances, the data received may be related to a subject lifting an object (e.g., picking up, setting down, raising, lowering, and/or otherwise moving the object). In one example, the data may be and/or may include video data of a subject performing a lift of an object, but this is not required. Alternatively or additionally, the data received may be data from sensors indicative of movement of body parts of the subject before, while, and/or after the subject is lifting the object and/or other suitable data associated with a lift of an object by the subject.

Based on the received data, values of one or more parameters related to a subject performing a task may be determined and monitored. For example, body movements of the subject performing the lift of an object maybe monitored and/or tracked 120, using the data, during the lift (e.g., over time). In some cases, computer vision techniques (e.g., as discussed herein) may be utilized to identify and track features of the subject while the subject moves during the lift of the object. Other techniques for monitoring and/or tracking movement of the subject while the subject is lifting the object are contemplated.

Tracking and/or monitoring body movements of the subject while the subject lifts the object may include determining one or more features related to movement of the subject during the lift. Example features related to movement of the subject during the lift include, but are not limited to, determining locations (e.g., coordinates) of one or more body parts, determining a posture of the subject, determining a trunk angle of the subject, determining a RWL using the RNLE, determining kinematics of the subject (e.g., determining velocity, acceleration, etc. of one or more body parts of the subject), determining a start and/or an end of the lift, and/or determining one or more other suitable features of or related to the subject.

Based on the body movements of the subject that are tracked and/or monitored while the subject is lifting the object, a value related to a load (e.g., weight) of the object lifted may be determined 130. In some cases, results of the tracking and/or monitoring 120 of body movements of the subject may be provided to a model of body movement relative to load lifted and the value related to the load of the object lifted may be outputted from the model. The following is an example formula for the model:

f(body movements during a lift of an object)=a load of the object (3)

The model may be any suitable model configured to determine or otherwise estimate a load of an object lifted by a subject and/or provide values related to the load of the object lifted. Although not required, the model may be a deep convolutional neural network trained to predict lifting loads using trajectories and/or kinematics (e.g., movements) of body parts of a subject performing the lift (e.g., trajectories and/or kinematics of body parts generated by the action of lifting an object). In some cases, the deep neural network model may be trained using videos of various subjects performing lifts of objects with known loads (e.g., weights) and/or trained in one or more other suitable manners. Other suitable models are contemplated.

In some cases, a general model for estimating loads of objects lifted by a subject may be calibrated for a specific individual. In one example, a deep neural network model and/or other suitable models may be calibrated to a specific subject by training the model, which may be initially developed using videos of various subjects lifting objects of known loads, with videos of the specific subject performing lifts of objects with known loads (e.g., weights) and/or training the model in one or more other suitable manners. A calibrated, subject specific model may facilitate providing values related to a load of the object lifted by the specific subject that may be percent values of a maximum load the specific subject is able to lift and/or facilitate providing more accurate values for the subject related to the load of the object lifted than would be possible with a general model for estimating loads lifted.

Values related to the load of the object may be any suitable value. Example values may be a value of the load (e.g., an absolute load or the weight) of the object lifted, an estimate of the load of the object lifted, a percent value indicative of the load of the object lifted being a percent of a maximum load associated with the subject (e.g., a percent value of the load relative to a maximum strength of the subject), a category indicative of a relative value of the load of the object (e.g., light/medium/heavy, below RWL/above RWL, etc.), a value of the Lifting Index (LI), etc.

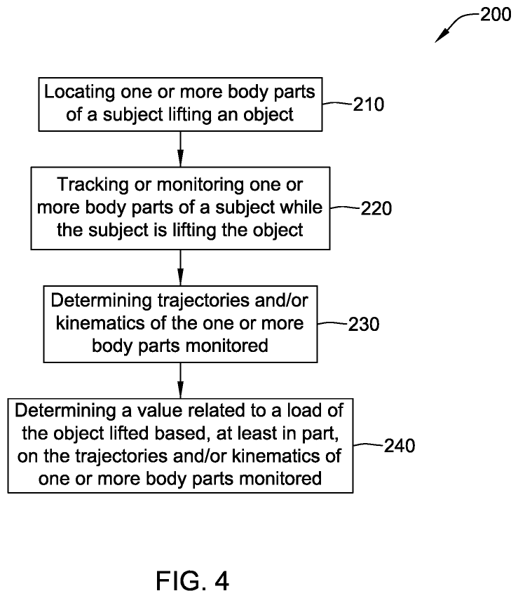

FIG. 4 depicts a schematic overview of an approach 200 for locating and/or tracking body parts of a subject to determine a load of an object lifted (e.g., picked up, set down, raised, lowered, or otherwise moved) by the subject. The approach 200 may be implemented using the monitoring or tracking system 10, where instructions to perform the elements of approach 200 may be stored in the memory 18 and executed by the processor 16. Additionally or alternatively, other suitable monitoring and/or tracking systems may be utilized to implement the approach 200.

The method 200 may include locating 210, using data (e.g., video data, sensor data, etc.) obtained from observing a subject lift an object, one or more body parts of the subject lifting the object. Example body parts of the subject that may be located and/or tracked may include, but are not limited to, a head, a left shoulder, a right shoulder, a left elbow, a right elbow, a left wrist, a right wrist, a left hand, a right hand, a left hip, a right hip, a left knee, a right knee, a left ankle, a right ankle, a left foot, a right foot, a trunk of the subject, etc. In one example, a head, a left shoulder, a right shoulder, a left elbow, a right elbow, a left wrist, a right wrist, a left hip, a right hip, a left knee, a right knee, a left ankle, and a right ankle of a subject may be located using the obtained data. In another example, a left wrist, a right wrist, a left elbow, a right elbow, a left shoulder, a right shoulder, a left hip, and a right hip of a subject may be located using the obtained data. Other examples are contemplated.

Further, in some cases, facial movements or expressions and/or other reactions (e.g. heart rate, etc.) to lifting an object may be identified in addition to or as an alternative to locating body parts of the subject lifting the object. Although locating body parts of the subject lifting the object may include locating body parts on a face or other portion of the subject, locating the body parts of the subject lifting the object may or may not include determining facial expressions of the subject lifting the object.

When the data obtained from observing a subject lift the object includes video data, computer vision techniques may be utilized to locate one or more body parts of the subject. In one example computer vision technique, a bounding box may be applied around a subject in video of the subject performing a lift of an object and the bounding box may be utilized to facilitate locating body parts and/or linkages of the subject. For example, a computer vision algorithm may receive an image or frames of video and a bounding box around a subject in the image or frames of video and output 2D coordinates of body parts (e.g., the example body parts discussed above and/or other suitable body parts). Application of a bounding box to and/or around a subject may be performed automatically via software (e.g., instructions stored in the memory 18 and/or other suitable memory) and/or may be performed manually by a user interacting with a user interface (e.g., the user interface 26 and/or other suitable user interface). Although other techniques are contemplated, example techniques for applying bounding boxes around subjects in video data, locating body parts and/or linkages (e.g., based on the bounding boxes around subjects), and tracking body parts and/or linkages (e.g., tracking body movements) over time are discussed in the US patent applications incorporated by reference herein, along with Ren, S., He, K., Girshick, R., & Sun, J. (2015), “Faster R-CNN: Towards real-time object detection with region proposal networks”, In Proceedings of advances in neural information processing systems (NeurIPS) (pp. 91-99), which is hereby incorporated by reference in its entirety for any and all purposes; and Xiao, B., Wu, H., & Wei, Y. (2018), “Simple baselines for human pose estimation and tracking”, In Proceedings of the european conference on computer vision (ECCV) (pp. 466-481), which is incorporated by reference herein in its entirety for any and all purposes.

The method 200 may further include tracking and/or monitoring 220 one or more of the located body parts (e.g., tracking and/or monitoring body movements) of the subject over time while the subject is lifting the object. In an example in which data related to the subject lifting the object is 2D video data from a camera perpendicular to a sagittal plane of a subject lifting the object, seven body parts of the subject and/or other suitable number of body parts may be located and tracked or monitored during the lift. Although other body parts and/or linkages may be utilized, the seven body parts tracked may be the head, along with a shoulder, an elbow, a wrist, a hip, a knee, and an ankle on a side of the subject facing the camera. Body parts (e.g., a left wrist) of the subject on a side (e.g., a left side) of the subject not facing the camera may be considered to have similar or identical positions during the lift as similar features (e.g., a right wrist) on a side (e.g., the right side) of the subject facing the camera, but this is not required. When data for similar body parts on both sides (e.g., a left side and a right side) of a subject is available, the left and right body parts may be tracked and/or monitored. In one example, a head, a left shoulder, a right shoulder, a left elbow, a right elbow, a left wrist, a right wrist, a left hip, a right hip, a left knee, a right knee, a left ankle, a right ankle, and/or other body parts of a subject may be tracked and/or monitored using the obtained data.

Further, in some cases, facial movements or expressions and/or other reactions (e.g. heart rate, etc.) to lifting an object may be monitored as part of, in addition to, or as an alternative to monitoring body parts of the subject lifting the object. Although monitoring body parts of the subject lifting the object may include monitoring body parts on a face or other portion of the subject, monitoring the body parts of the subject lifting the object may or may not include monitoring facial expressions of the subject lifting the object.

In some case, the tracking and/or monitoring 220 of body parts of the subject while the subject is lifting the object may include tracking coordinates of one or more body parts of the located body parts of the subject while the subject is lifting the object. In some cases, tracking coordinates of the one or more body parts while the subject is lifting the object may include identifying coordinates of one or more body parts of the subject at one or more time instances (e.g., sampling or time increments) during the lift of the object. Example time instances may include, but are not limited to, a partial second, a second, five seconds, ten seconds, etc. In some cases, time increments may be predetermined, automatically set by a computer vision system based on a length of the lift, a default value, a frame rate of the video, and/or other suitable increments manually or automatically set.

In one example of tracking body parts, coordinates of body parts in frames of video may be identified as discussed above with respect to the locating 210 body parts in the method 200 and then a computer vision algorithm (e.g., the computer vision algorithm that locates coordinates of body parts and/or a different suitable computer vision algorithm) may determine motion information and track the located body parts from coordinates in successive frames. Such an example technique is discussed in Xiao, B. et al., (2018), “Simple baselines for human pose estimation and tracking”, In Proceedings of the European conference on computer vision (ECCV), which is incorporated by reference above.

The tracking and/or monitoring 220 of body parts of the subject while the subject is lifting the object may result in determining one or more feature related to movement of the subject during the lift. Similar to as discussed above, example features related to movement of the subject during the lift include, but are not limited to, determining locations (e.g., coordinates) of one or more body parts, determining trajectories of one or more body parts, determining a posture of the subject, determining a trunk angle of the subject, determining a RWL using the RNLE, determining kinematics of the subject (e.g., determining velocity, acceleration, etc. of one or more body parts of the subject), determining a start and/or an end of the lift, and/or one or more other suitable features of or related to the subject.

The method 200 may further include determining 230 trajectories and/or kinematics of or related to the one or more body parts tracked and/or monitored. Example trajectories and/or kinematics of or related to the one or more body parts tracked and/or monitored may include, but are not limited to, trajectories and/or kinematics of body part position, velocities of body parts, acceleration of body parts, etc.

In some cases, the determined coordinates of the monitored and/or tracked one or more body parts while the subject lifts the object may be utilized to determine trajectories and/or kinematics associated with the one or more body parts. In one example, trajectories of a body part may be considered to be a slope of coordinates of the body part between a predetermined number of time instances (e.g., a slope between consecutive time instances, a slope between every third time instance, and/or other suitable time instances), an average slope during a lift, and/or one or more other suitable trajectory values of a body part. In another example, kinematics of a body part may be velocity and/or acceleration of the body part, which may be determined from changes in coordinate locations of the body part at different time instances and a sum of the time between the different time instances. Other suitable techniques for determining trajectories and/or kinematics of the subject (e.g., one or more body parts of the subject) lifting the object are contemplated.

An example technique for determining trajectories and/or kinematics of or related to the one or more body parts tracked and/or monitored may use the coordinates of each body part at each frame, or a desired number of frames, of video of a subject performing a lift and then link the coordinates of each body part into an initial trajectory. The initial trajectories for each of the body parts may be smoothed using motion tracking algorithms (e.g., the computer vision algorithm(s) used to locate and/or track body parts and/or a different suitable computer vision algorithm). In some cases, an optical flow method may be used for tracking the motion of each pixel between two video frames to determine trajectories and/or kinematics of or related to tracked and/or monitored body parts. An example optical flow method is described in Sun, D., Yang, X., Liu, M. Y., & Kautz, J., (2018), “Pwc-net: Cnns for optical flow using pyramid, warping, and cost volume”, In Proceedings of the IEEE conference on computer vision and pattern recognition (pp. 8934-8943), which is incorporated by reference herein in its entirety for any and all purposes.

Based, at least in part, on the determined trajectories and/or kinematics of the one or more body parts of the subject that are tracked and/or monitored while the subject is lifting the object, a value related to a load (e.g., weight) of the object lifted may be determined 240. In some cases, the determined trajectories and/or kinematics of the one or more body parts of the subject lifting the object may be provided to a model of body movement relative to load lifted and the value related to the load of the lifted object may be outputted from the model. As discussed above, equation (3) is an example formulation of the model and the following is an additional or alternative example formula for the model:

f(movements of one or more body parts during a lift of an object)=a value related to a load of the object (4)

The model may be any suitable model configured to determine or otherwise estimate a load or a value related to a load of an object lifted by a subject. Similar to as discussed above with respect to the method 100 and although not required, the model may be a deep convolutional neural network model trained to predict lifting loads using trajectories and/or kinematics of body parts of a subject performing the lift. In some cases, the deep neural network model may be trained using videos of various subjects performing lifts of objects with known loads (e.g., weights) and/or trained in one or more other suitable manners. Other suitable models are contemplated.

Further, and similar to as discussed above, the deep neural network model and/or other suitable models may be calibrated to a specific subject by training the model, which may be initially developed using data (e.g., videos and/or other suitable data) of various subjects lifting objects of known loads, with data of the specific subject performing lifts of objects with known loads (e.g., weights) and/or training the model in one or more other suitable manners. A calibrated subject specific model may facilitate providing values related to a load of the object lifted by the specific subject that may be percent values of a maximum load the specific subject is able to lift (e.g., a percent of a total exertion) and/or facilitate providing more accurate values for the subject related to the load of the object lifted.

As discussed herein, values related to the load of the object lifted may be any suitable value. Example values may be a value of the load (e.g., the weight) of the object lifted, a percent value indicative of the load of the object lifted being a percent of a maximum load associated with the subject (e.g., a percent of a maximum strength of the subject), a category indicative of a relative load of the object (e.g., light/medium/heavy, below RWL/above RWL, etc.), a value of the Lifting Index (LI), etc.

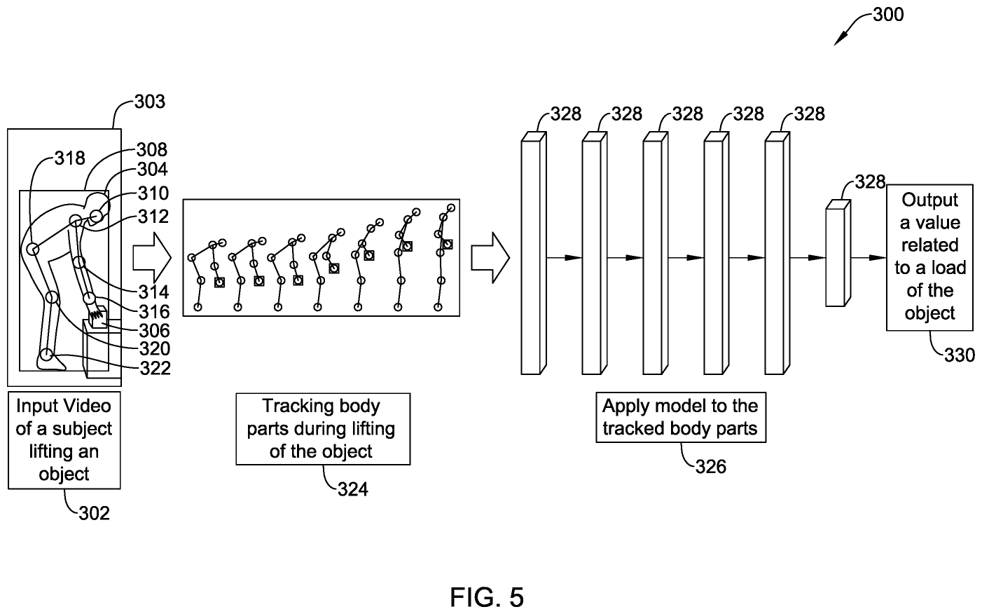

FIG. 5 is a schematic diagram of an illustrative technique 300 for determining a value related to a load of an object lifted by a subject based on inputted video data 302 of a subject 304 lifting an object 306. The video may be any suitable type of video including, but not limited to, 2D video, 3D video, virtual reality video, and/or other suitable types of video. In the example depicted in FIG. 5, the inputted video is 2D video taken perpendicular to a subject's sagittal plane.

In the technique 300, the subject 304 may be identified in a frame 303 of the inputted video. In some cases, a bounding box 308 may be applied around (e.g., tightly around or otherwise around) the subject 304 lifting an object 306 to facilitate identifying the subject 304 in various frames 303 of the inputted video, identifying body parts and/or locations of body parts, identifying postures of the subject 304, identifying a beginning and/or ending of a lift, etc. The body parts of the subject 304 that are identified in the frame 303 of video depicted in FIG. 5 include a head 310, a shoulder 312, an elbow 314, a wrist 316, a hip 318, a knee 320, and an ankle 322, which are shown linked together with lines to facilitate tracking over the frames of inputted video.

Further the technique 300 may include monitoring or tracking 324 body parts of the subject 304 while the subject 304 lifts the object 306. Tracking of the body parts of the subject 304 may be done in any manner discussed or incorporated herein and/or in other suitable manners. As depicted in FIG. 5, a computer vision algorithm may be configured to track body part locations (e.g., coordinates) and/or linkages over the frames of the inputted video of the subject 304 lifting the object 306.

Once the body parts of the subject 304 have been identified and/or located, the locations of the body parts and/or values based on the locations of the body parts may be provided to a model and the model may be applied 326 to the tracked locations of the body parts. The model may be trained or otherwise configured to relate trajectories and/or kinematics of body parts to values related to a load of an object lifted by a subject. In one example, the model may map or otherwise associate 2D trajectories and/or kinematics of body parts (e.g., position, velocity, and acceleration, and/or other suitable trajectories and/or kinematic) into the value related to the load of the object lifted by the subject.

The model may be a model of the type discussed herein and/or other suitable type of model and may have one or more layers 328. In one example, the model may be a 1D convolutional neural network model having five layers 328 that may be configured to relate body movements during a lift of an object to a load (e.g., weight) of the object. In another example, the model configured to relate body movements during a lift of an object to a load of the object may be a deep neural network model having two layers 328 being convolutional layers with nonlinear activation functions (e.g., rectified linear units) in-between, followed by three layers 328 being transformer layers, which may be followed by one layer 328 being a fully connected layer. When the model includes one or more transformer layers, positions that encode time steps of tracked body parts or linkages may be added to an input of the first transformer layer. Other suitable configurations of neural network models and/or other models configured to relate body movements during a lift of an object to a load of the object are contemplated.

Example neural network models including convolutional layers are discussed in LeCun, Y., & Bengio, Y. (1995), “Convolutional networks for images, speech, and time series”, The handbook of brain theory and neural networks, 3361(10), which is hereby incorporated by references in its entirety for any and all purposes. Example neural network models including nonlinear activation functions are discussed in Krizhevsky, A., Sutskever, I., & Hinton, G. E. (2012), “Imagenet classification with deep convolutional neural networks”, Advances in neural information processing systems, 25, 1097-1105, which is hereby incorporated by references in its entirety for any and all purposes.

In the technique 300, the model may output 330 a value related to a load of the object 306 lifted by the subject 304 based on body movements of the subject 304. As discussed herein, the value related to a load of the object 306 may be a direct estimate of the load (e.g., an estimate of the weight of the load), a relative value relating the load to another value (e.g., a percentage value of a maximum lifting weight for a subject, a value of the LI, etc.), a category relating the load to other loads (e.g., light/medium/heavy, etc.), and/or other suitable values. When the value related to a load of the object 306 is a direct estimate of the load, the value may be provided for use in a further computing process in combination with a determined RWL to determine a LI value for the lift of an object performed by the subject 304. Such use of the determined value related to the object 306 removes the requirement to manually weigh every object 306 lifted by the subject 304 and facilitates providing a monitoring and/or tracking system capable of providing real time LI values during lifts by the subject 304.

Although the monitoring or tracking system 10 is discussed in view of manual lifting tasks, similar disclosed concepts may be utilized for other tasks involving movement. Example tasks may include, but are not limited to, manual lifting, sorting, typing, performing surgery, throwing a ball, weight lifting, etc. Additionally, the concepts disclosed herein may apply to analyzing movement of people, other animals, machines, and/or other devices.

Further discussion of monitoring or tracking systems, techniques utilized for processing data, and performing assessments (e.g., injury risk assessments) is found in U.S. Patent Application Publication Number 2019/0012794 A116 filed on Oct. 6, 2017, and is titled MOVEMENT MONITORING SYSTEM, which is hereby incorporated by reference in its entirety for any and all purposes, and U.S. Patent Application Publication Number 2019/0012531 A1 filed on Jul. 18, 2018, and is titled MOVEMENT MONITORING SYSTEM, which is hereby incorporated by reference in its entirety for any and all purposes.

Those skilled in the art will recognize that the present disclosure may be manifested in a variety of forms other than the specific embodiments described and contemplated herein. Accordingly, departure in form and detail may be made without departing from the scope and spirit of the present disclosure as described in the appended claims.

what is claimed is:

1. A subject monitoring system comprising:
an input port for receiving data related to a subject lifting an object;
a controller in communication with the input port, the controller is configured to:
monitor body movements of the subject lifting the object using the data; and
determine a value related to a load of the object based on the body movements monitored.

2. The system of claim 1, wherein the controller is further configured to:
locate a plurality of body parts of the subject using the data; and
wherein the monitoring body movements of the subject includes identifying coordinates of one or more body parts of the plurality of body parts located at one or more time instances while the subject is lifting the object.

3. The system of claim 2, wherein the plurality of body parts of the subject include two or more of the following body parts: a head of the subject, a left shoulder of the subject, a right shoulder of the subject, a left elbow of the subject, a right elbow of the subject, a left wrist of the subject, a right wrist of the subject, a left hip of the subject, a right hip of the subject, a left knee of the subject, a right knee of the subject, a left ankle of the subject, and a right ankle of the subject.

4. The system of claim 1, wherein the monitoring body movements of the subject includes determining body kinematics of the subject lifting the object.

5. The system of claim 4, wherein the determined body kinematics of the subject include one or both of a velocity of a body part of the subject while the subject is lifting the object and an acceleration of a body part of the subject while the subject is lifting the object.

6. The system of claim 1, wherein the monitoring body movements of the subject includes determining a posture of the subject while the subject is lifting the object.

7. The system of claim 1, wherein the value related to a load of the object based on the body movements monitored is an estimated value of the load of the object.

8. The system of claim 1, wherein the value related to a load of the object based on the body movements monitored is a category indicative of a relative value of the load of the object.

9. The system of claim 1, wherein the value related to a load of the object based on the body movements monitored is a lifting index value, the lifting index value is a load of the object estimated based on the body movements monitored divided by a recommended weight limit (RWL) for the subject lifting the object.

10. The system of claim 9, the controller is further configured to determine the RWL based on the data.

11. The system of claim 1, the controller is further configured to:
train a neural network model of the subject using the data; and
wherein the value related to a load of the object is determined based on the neural network model of the subject and the body movements monitored.

12. A computer readable medium having stored thereon in a non-transitory state program code for use by a computing device, the program code causing the computing device to execute a method for determining a value related to a load of an object lifted by a subject, the method comprising:
locating one or more body parts of the subject using data related to the subject lifting the object;
monitoring one or more body parts of the subject while the subject is lifting the object; and
determining a value related to a load of the object based on the one or more body parts monitoring.

13. The computer readable medium of claim 12, wherein the monitoring of the one or more body parts of the subject includes determining body kinematics of the subject lifting the object.

14. The computer readable medium of claim 12, wherein the monitoring the one or more body parts of the subject includes determining a posture of the subject.

15. The computer readable medium of claim 12, wherein the determining the value related to the load of the object includes determining an estimate of the load of the object based on the body movements monitored.

16. The computer readable medium of claim 15, the method further comprising:
determining a recommended weight limit (RWL) for the subject lifting the object using the data related to the subject lifting the object; and
wherein the determining the value related to the load of the object includes determining a value of a lifting index using the estimate of the load of the object and the RWL.

17. A method of determining a load of an object lifted by a subject, the method comprising:
monitoring one or more body parts of a subject while the subject is lifting an object;
determining kinematics of at least one body part of the subject while the subject is lifting the object based on the monitoring of the one or more body parts of the subject; and
determining a value related to a load of the object based on the determined kinematics of the at least one body part.

18. The method of claim 17, further comprising:
determining a posture of the subject lifting the object based on the monitoring of the one or more body parts of the subject.

19. The method of claim 17, wherein the determining the value related to the load of the object includes determining an estimate of the load of the object.

20. The method of claim 17, wherein the monitoring of the one or more body parts of the subject includes applying a bounding box around the subject in a video of the subject lifting the object.
